(19) United States
(12) Patent Application Publication
(10) Pub. No.: US 2008/0014579 A1
(43) Pub. Date: Jan. 17, 2008

(54) Gene expression profiling in colon cancers

(75) Inventors: Chunmei Liu, Cupertino, CA (US); John Palma, San Ramon, CA (US); Janet A. Warrington, Los Altos, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 10/777,860

(22) Filed: Feb. 11, 2004

Related U.S. Application Data
(60) Provisional application No. 60/446,893, filed on Feb. 11, 2003.

(51) Int. Cl. C12Q 1/68; C40B 20/04; C40B 30/00

(52) U.S. Cl. 435/6; 506/4; 506/7

(57) ABSTRACT

Sets of genetic markers for specific tumor classes are described, as well as methods of identifying a biological sample based on these markers. Genes that are differentially expressed between different stages of colorectal tumors are identified. Genes that are differentially expressed between tumors that have metastasized to the regional lymph nodes and tumors that have not metastasized to the regional lymph nodes are identified. Changes in gene expression pattern between two samples or between a sample and a control may be used to determine tumor stage. Also described are diagnostic, prognostic, and therapeutic screening uses for these markers, as well as oligonucleotide arrays comprising these markers.

### RELATED APPLICATIONS

The present application claims priority to U.S. Provisional Application No. 60/446,893 filed Feb. 11, 2003, the disclosure of which is incorporated herein by reference in its entirety.

### FIELD OF THE INVENTION

The methods of the invention relate generally to genes that are differentially expressed between different stage colorectal tumors and methods of using the same.

### REFERENCE TO SEQUENCE LISTING

The Sequence Listing submitted on compact disk is hereby incorporated by reference. The file on the disk is named 3581seq1.txt, the file is 137 MB and the date of creation is Feb. 11, 2004.

### BACKGROUND

Many cellular events and processes are characterized by altered expression levels of one or more genes. Differences in gene expression correlate with many physiological processes such as cell cycle progression, cell differentiation and cell death. Changes in gene expression patterns also correlate with changes in disease or pharmacological state. For example, the lack of sufficient expression of functional tumor suppressor genes and/or the over expression of oncogene/protooncogenes could lead to tumorgenesis (Marshall, Cell, 64:313-326 (1991); Weinberg, Science, 254:1138-1146 (1991), incorporated herein by reference for all purposes). Thus, changes in the expression levels of particular genes (e.g. oncogenes or tumor suppressors) serve as signposts for different physiological, pharmacological and disease states.

Classification of biological samples from individuals is not an exact science. In many instances, accurate diagnosis and safe and effective treatment of a disorder depend on being able to discern biological distinctions among cell or tissue samples such as the stage of a tumor. The classification of a sample from an individual into particular disease classes has often proven to be difficult, incorrect, or equivocal. Typically, using traditional methods such as histochemical analysis, immunophenotyping, and cytogenetic analysis, only one or two characteristics of the sample are analyzed to determine the sample's classification. Inaccurate results can lead to incorrect diagnoses and potentially ineffective or harmful treatment. Thus a need exists for an accurate and efficient method for identifying tumor types and differentiating between different stages of tumors.

### SUMMARY OF THE INVENTION

The present invention is a method to analyze colorectal tumor samples to differentiate between tumors that have metastasized from tumors that have not metastasized using gene expression profiles. In one embodiment tumors that have invaded the lymph nodes are distinguished from tumors that have not (Dukes' C versus Dukes' B) based on gene expression pattern. A collection of genes that have been identified as being differentially expressed in Dukes' C versus Dukes' B stage tumors is disclosed. The genes may be used individually or in groups of 2, 5, 10, 25, 50 or more to analyze tissues of unknown state. For example, the genes may be used to stage tumors, to predict treatment outcome, to assist in the design of new drugs or treatment regimens or as drug targets.

In one embodiment a method of classifying a colorectal tumor is disclosed. The method comprises obtaining a sample of cells derived from a colorectal tumor; isolating a gene expression product from at least one informative gene from one or more cells in the sample; determining a gene expression profile of at least one informative gene in the sample; and comparing the gene expression profile of the at least one informative gene in the sample to the gene expression profile of the at least one informative gene in at least one sample of known classification wherein the gene expression profile is correlated with a specific colorectal tumor type. The tumor type may be selected from the group consisting of Dukes' A, B, C or D stage colorectal tumors. The tumor may or may not have regional lymph node metastasis. The expression product may be total RNA, mRNA or proteins. The gene expression profile may be determined using an array of nucleic acid probes. Informative genes may be selected from the group consisting of the genes in Tables 2-3.

In one embodiment a method of identifying compounds that inhibit or promote metastasis of a colorectal tumor to the regional lymph nodes is disclosed. The method comprises obtaining samples of cells from a colorectal tumor before and after treatment; isolating a gene expression product from the samples and determining an expression profile for at least one gene in Tables 2 and 3; comparing the direction of change between the samples to the direction of change in Tables 2 and 3 and identifying the compound as a promoter of metastasis if the direction of change is the same as the direction of change for that at least one informative gene from Dukes' B to Dukes' C in Tables 2 and 3 or as an inhibitor of metastasis if the direction of change is the opposite of the direction of change in Tables 2 and 3.

In another embodiment gene expression profiles of the genes in Tables 2 and 3 are used to predicting the efficacy of a compound for treating a colorectal tumor. Samples are collected before and after treatment with the compound and the expression profile for at least one gene in Tables 2 and 3 is determined. The change in the expression profile from the treated to the untreated is compared to the change in expression profile between Dukes' B and Dukes' C stage tumors, for example, using Tables 2-3. The direction of change, up or down, is compared.

In another embodiment a method for classifying malignant colon cells is disclosed. In another embodiment a colorectal tumor is classified as being positive or negative for regional lymph node metastases based on the information in Tables 2-3 and comparison of expression profiles.

### DETAILED DESCRIPTION

### A. General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. No. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. No. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80:1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. patent application Ser. No. 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. patent application Ser. No. 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. patent application Ser. Nos. 10/063,559, 60/349,546, 60/376,003, 60/394,574, 60/403,381.

A nucleic acid sample may be obtained by any method known in the art. One of skill in the art will appreciate that it is desirable to have nucleic samples containing target nucleic acid sequences that reflect the transcripts of interest. Therefore, suitable nucleic acid samples may contain transcripts of interest. Suitable nucleic acid samples, however, may contain nucleic acids derived from the transcripts of interest. As used herein, a nucleic acid derived from a transcript refers to a nucleic acid for whose synthesis the mRNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable samples include, but are not limited to, transcripts of the gene or genes, cDNA reverse transcribed from the transcript, cRNA transcribed from the cDNA, DNA amplified from the genes, RNA transcribed from amplified DNA, and the like. Transcripts, as used herein, may include, but not limited to pre-mRNA nascent transcript(s), transcript processing intermediates, mature mRNA(s) and degradation products. It is not necessary to monitor all types of transcripts to practice this invention. For example, one may choose to practice the invention to measure the mature mRNA levels only.

In one embodiment, such sample is a homogenate of cells or tissues or other biological samples. Preferably, such sample is a total RNA preparation of a biological sample. More preferably in some embodiments, such a nucleic acid sample is the total mRNA isolated from a biological sample. Those of skill in the art will appreciate that the total mRNA prepared with most methods includes not only the mature mRNA, but also the RNA processing intermediates and nascent pre-mRNA transcripts. For example, total mRNA purified with poly (T) column contains RNA molecules with poly (A) tails. Those poly A+RNA molecules could be mature mRNA, RNA processing intermediates, nascent transcripts or degradation intermediates.

Biological samples may be of any biological tissue or fluid or cells. Frequently the sample will be a “clinical sample” which is a sample derived from a patient. Clinical samples provide rich sources of information regarding the various states of genetic network or gene expression. Some embodiments of the invention are employed to detect mutations and to identify the function of mutations. Such embodiments have extensive applications in clinical diagnostics and clinical studies. Typical clinical samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes.

Another typical source of biological samples is cell cultures where gene expression states can be manipulated to explore the relationship among genes. In one aspect of the invention, methods are provided to generate biological samples reflecting a wide variety of states of the genetic network.

One of skill in the art would appreciate that in some embodiments it is desirable to inhibit or destroy RNase present in homogenates before homogenates can be used for hybridization. Methods of inhibiting or destroying nucleases are well known in the art. In some preferred embodiments, cells or tissues are homogenized in the presence of chaotropic agents to inhibit nuclease. In some other embodiments, RNases are inhibited or destroyed by heat treatment followed by proteinase treatment.

Methods of isolating total mRNA are also well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) which is incorporated herein by reference.

In a preferred embodiment, the total RNA is isolated from a given sample using, for example, an acid guanidinium-phenol-chloroform extraction method and polyA⁺ mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)). See also PCT/US99/25200 for complexity management and other sample preparation techniques, which is hereby incorporated by reference in its entirety.

Frequently, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids to achieve quantitative amplification.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high density array may then include probes specific to the internal standard for quantification of the amplified nucleic acid.

Cell lysates or tissue homogenates often contain a number of inhibitors of polymerase activity. Therefore, RT-PCR typically incorporates preliminary steps to isolate total RNA or mRNA for subsequent use as an amplification template. One tube mRNA capture methods may be used to prepare poly(A)+ RNA samples suitable for immediate RT-PCR in the same tube (Boehringer Mannheim). The captured mRNA can be directly subjected to RT-PCR by adding a reverse transcription mix and, subsequently, a PCR mix. In a particularly preferred embodiment, the sample mRNA is reverse transcribed with a reverse transcriptase and a primer consisting of oligo dT and a sequence encoding the phage T7 promoter to provide single stranded DNA template. The second DNA strand is polymerized using a DNA polymerase. After synthesis of double-stranded cDNA, T7 RNA polymerase is added and RNA is transcribed from the cDNA template. Successive rounds of transcription from each single cDNA template result in amplified RNA. Methods of in vitro polymerization are well known to those of skill in the art (see, e.g., Sambrook, supra).

It will be appreciated by one of skill in the art that the direct transcription method described above provides an antisense (aRNA) pool. Where antisense RNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes may be of either sense as the target nucleic acids include both sense and antisense strands.

The protocols cited above include methods of generating pools of either sense or antisense nucleic acids. Indeed, one approach can be used to generate either sense or antisense nucleic acids as desired. For example, the cDNA can be directionally cloned into a vector (e.g., Stratagene's p Bluscript II KS (+) phagemid) such that it is flanked by the T3 and T7 promoters. In vitro transcription with the T3 polymerase will produce RNA of one sense (the sense depending on the orientation of the insert), while in vitro transcription with the T7 polymerase will produce RNA having the opposite sense. Other suitable cloning systems include phage lambda vectors designed for Cre-loxP plasmid subcloning (see e.g., Palazzolo et al., Gene, 88: 25-36 (1990)).

Other analysis methods that can be used in the present invention include electrochemical denaturation of double stranded nucleic acids, U.S. Pat. No. 6,045,996 and 6,033,850, the use of multiple arrays (arrays of arrays), U.S. Pat. No. 5,874,219, the use of scanners to read the arrays, U.S. Pat. Nos. 5,631,734; 5,744,305; 5,981,956 and 6,025,601, methods for mixing fluids, U.S. Pat. No. 6,050,719, integrated device for reactions, U.S. Pat. No. 6,043,080, integrated nucleic acid diagnostic device, U.S. Pat. No. 5,922,591, and nucleic acid affinity columns, U.S. Pat. No. 6,013,440. All of the above patents are hereby incorporated by reference in their entireties.

### B. Definitions

An array comprises a solid support with peptide or nucleic acid probes attached to the support. Arrays typically comprise a plurality of different nucleic acid or peptide probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., Science, 251:767-777 (1991). Each of which is incorporated by reference in its entirety for all purposes. These arrays may generally be produced using mechanical synthesis methods or light directed synthesis methods which incorporate a combination of photolithographic methods and solid phase synthesis methods. Techniques for the synthesis of these arrays using mechanical synthesis methods are described in, e.g., U.S. Pat. No. 5,384,261, incorporated herein by reference in its entirety for all purposes. Although a planar array surface is preferred, the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes. Arrays may be packaged in such a manner as to allow for diagnostics or other manipulation of an all inclusive device, see for example, U.S. Pat. Nos. 5,856,174 and 5,922,591 incorporated in their entirety by reference for all purposes. See also U.S. patent application Ser. No. 09/545,207, filed Apr. 7, 2000 for additional information concerning arrays, their manufacture, and their characteristics. It is hereby incorporated by reference in its entirety for all purposes.

A sample may or may not be affected with a disease state. According to the present invention, a disease state or disease status refers to any abnormal biological state of a cell. This includes but is not limited to an interruption, cessation or disorder of body functions, systems or organs. In general, a disease state will be detrimental to a biological system. With respect to the present invention, any biological state, such as a premalignancy state or malignancy state that is associated with a disease or disorder is considered to be a disease state. A pathological state is the equivalent of a disease state.

Disease states can be further categorized into different levels of disease state. As used in the present invention, the level of a disease or disease state is a measure reflecting the progression of a disease or disease state. Generally, a disease or disease state will progress through a plurality of levels or stages, wherein the affects of the disease become increasingly severe, for example, Dukes' A, B, C or D stage. A disease state may be determined by a variety of methods including any method known in the art. Disease state may be determined for example by histological analysis of the affected tissue, by the presence or absence of one or more gene product or by the expression pattern of one or more genes.

In order to alleviate or alter a disease state, a therapy or therapeutic regimen is often undertaken. A therapy or therapeutic regimen, as used herein, refers to a course of treatment intended to reduce or eliminate the affects or symptoms of a disease or to prevent progression of a disease from one state to a second more detrimental state. A therapeutic regimen will typically comprise, but is not limited to, a prescribed dosage of one or more drugs, surgery or radiation treatment. Therapies, ideally, will be beneficial and reduce the disease state but in many instances the effect of a therapy will have non-desirable effects as well. The effect of therapy will also be impacted by the physiological state of the sample. The genotype of the patient may also impact the side effects and efficacy of a selected therapy. Genotype may be used to determine which therapy or therapeutic regimen is likely to me most effective.

Treatment with drugs may affect the pharmacological state of a sample. The pharmacological state or pharmacological status of a sample relates to changes in the biological status following drug treatment. Some of the changes following drug treatment or surgery may be relevant to the disease state. Some may be unrelated-side effects of the therapy. Some will be specific to physiological state. Indicators of pharmacological state include, but are not limited to, duration of therapy, types and doses of drugs prescribed, degree of compliance with a given course of therapy, and/or unprescribed drugs ingested.

One measurement of cellular constituents that is particularly useful in the present invention is the expression profile or gene expression profile. As used herein, an expression profile comprises measurement of the relative abundance of a plurality of cellular constituents. Such measurements may include RNA or protein abundances or activity levels. The expression level of a gene is the abundance of the mRNA of that gene from a sample. The expression level may be a normalized value. The expression profile can be a measurement for example of the transcriptional state or the translational state of two or more genes. See U.S. Pat. Nos. 6,040,138, 5,800,992, 6,020135, 6,033,860 and U.S. Ser. No. 09/341,302 which are hereby incorporated by reference in their entireties. See also Sharan et al. Ernst Schering Res Found Workshop. 2002;(38):83-108 which is incorporated herein by reference in its entirety. A gene expression profile may include expression levels of genes that are not informative, as well as informative genes. Phenotype classification can be made by comparing the gene expression profile of the sample with respect to one or more informative genes with one or more gene expression profiles (e.g., in a database). Using the methods described herein, expression of numerous genes can be measured simultaneously. The assessment of numerous genes provides for a more accurate evaluation of the sample because there are more genes that can assist in classifying the sample. A gene expression profile may involve only those genes that are increased in expression in a sample, only those genes that are decreased in expression in a sample, or a combination of genes that are increased and decreased in expression in a sample.

As used herein informative gene refers to a gene whose expression correlates with a particular phenotype. Expression profiles obtained for informative genes can be used to determine, for example, the presence or absence of a Dukes' C tumor or if a candidate compound increases or decreases gene expression in a sample. Samples can be classified according to their broad expression profile, or according to the expression levels of particular informative genes. The genes that are relevant for classification are referred to herein as “informative genes”. Not all informative genes for a particular class distinction must be assessed in order to classify a sample. Similarly, the set of informative genes that characterize one phenotypic effect may or may not be the same as the set of informative genes for a different phenotype effect. For example, a subset of the informative genes that demonstrate a high correlation with a class distinction can be used in classifying the presence of a Dukes' C tumor or predicting metastasis. This subset can be, for example, 1, 2, 3, 5, 10, 25, or 50 or more genes. Typically the accuracy of the classification increases with the number of informative genes that are assessed. Informative genes include but are not limited to the particular genes shown in Tables 2 and 3.

Direction of change is an indication of whether a gene is expressed at a higher or lower level in a first sample compared to a second sample. If a gene is expressed at a higher level in a first sample when compared to a second sample or collection of samples the direction of change is up indicating an increase in expression in the first sample relative to the second. If a gene is expressed at a lower level in a first sample when compared to a second sample or collection of samples the direction of change is down indicating a decrease in expression in the first sample relative to the second. In Tables 2-3 if the direction of change is up this indicates that the expression of that gene in the Dukes' C samples is increased relative to the expression of that gene in the Dukes' B samples. If the direction of change is down this indicates that the expression of that gene in the Dukes' C samples is decreased relative to the expression of that gene in the Dukes' B samples.

The magnitude of the change is the fold change. In Tables 2 and 3 fold change is expressed as the ratio of the expression level of the sample that is expressed at a higher level to the expression level of the sample that is expressed at a lower level. If the gene is up regulated in C compared to B the fold change number given is C/B if the gene is down regulated in C compared to B the fold change number given is B/C so that the magnitude of the difference can be compared.

The cellular constituent can be either up regulated in the experimental relative to the reference or down regulated in the experimental relative to the reference. Differential gene expression can also be used to distinguish between cell types, tissue types or nucleic acids. See U.S. Pat. Nos. 5,800,992, 6,020,153, 6,033,860, 6,171,798, 6,391,550, 6,548,257, and 6,576,424, which are each incorporated herein by reference in their entireties.

The gene expression value measured or assessed is a numeric value obtained from an apparatus that can measure gene expression levels which may be normalized. Gene expression levels refer to the amount of expression of the gene expression product. Such data is obtained, for example, from a GeneChip® probe array or microarray (Affymetrix, Inc.) and the expression levels are calculated with software. (See the GeneChip® Expression Analysis Technical Manual, Affymetrix, Inc. 2002, which is incorporated herein by reference in its entirety for all purposes).

The transcriptional state of a sample includes the identities and relative abundances of the RNA species, especially mRNAs present in the sample. Preferably, a substantial fraction of all constituent RNA species in the sample are measured, but at least, a sufficient fraction is measured to characterize the state of the sample. Transcriptional state can be conveniently determined by measuring transcript abundances by any of several existing gene expression technologies.

Translational state includes the identities and relative abundances of the constituent protein species in the sample. As is known to those of skill in the art, the transcriptional state and translational state are related.

The gene expression monitoring system, in a preferred embodiment, may comprise a nucleic acid probe array (such as those described above), membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are expressly incorporated herein by reference. The gene expression monitoring system may also comprise nucleic acid probes in solution. The gene expression monitoring system according to the present invention may be used to facilitate a comparative analysis of expression in different cells or tissues, different subpopulations of the same cells or tissues, different disease states of the same cells or tissue, different developmental stages of the same cells or tissue, or different cell populations of the same tissue. See U.S. Pat. No. 6,033,860 and U.S. patent application Ser. Nos. 09/102,167, 09/734,752 and 10/222,206.

Complementary or substantially complementary refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are the to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementary exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference. Effective amount refers to an amount sufficient to induce a desired result.

Genome is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term hybridization refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.”

Hybridization conditions will typically include salt concentrations of less than about 1M, more usually less than about 500 mM and preferably less than about 200 mM. Hybridization temperatures can be as low as 5 degree C., but are typically greater than 22 degree C., more typically greater than about 30 degree C., and preferably in excess of about 37 degree C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics. See U.S. Pat. No. 6,156,501 filed Apr. 3, 1996. Hybridizing specifically to refers to the binding, duplexing, or hybridizing of a molecule substantially to or only to a particular nucleotide sequence or sequences under stringent conditions when that sequence is present in a complex mixture (e.g., total cellular) DNA or RNA.

Isolated nucleic acid is an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

Mixed population or complex population refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population, but include other undesirable populations. For example, a complex population of nucleic acids may be a sample which has been enriched for desired messenger RNA (mRNA) sequences but still includes some undesired ribosomal RNA sequences (rRNA).

mRNA or mRNA transcripts as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

Nucleic acid library is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non- nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Nucleic acids according to the present invention may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An oligonucleotide or polynucleotide is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

A probe is a surface-immobilized molecule that can be recognized by a particular target. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Solid support, support, and substrate are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

A target is a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

Kurtosis in statistics is the degree of flatness or ‘peakedness’ in the region of mode of a frequency curve. It is measured relative to the ‘peakedness’ of the normal curve. It tells us the extent to which a distribution is more peaked or flat-topped than the normal curve. If the curve is more peaked than a normal curve it is called ‘Lepto Kurtic.’ In this case items are more clustered about the mode. If the curve is more flat-toped than the more normal curve, it is Platy-Kurtic. The normal curve itself is known as “Meso Kurtic.” Kurtosis is a measure of how “fat” a probability distribution's tails are, measured relative to a normal distribution having the same standard deviation.

Metastasis refers to the spread of cancer from its original site to other areas in the body. Cancer cells have the ability to invade the blood vessels and find their way into the bloodstream or lymph system. Once in the blood, cancer cells can go to virtually any part of the body and make a home for themselves. Each cancer has a particular way of spreading. The following formula can be used to calculate kurtosis:

$$kurtosis = \frac{\sum {( x - μ )}^{4}}{N {σ}^{4}} - 3$$

where s is the standard deviation. The kurtosis of a normal distribution is 0.

### C. Identification of Colorectal Cancer Metastases Expression Pattern and Candidate Genes

Colorectal cancer is the second leading cause of cancer-related death in the United States. Early detection of colon cancer in its premalignant stages prevents progression to invasive cancer. Available screening modalities include chemical testing for the presence of occult blood in the stool, endoscopic visualization of the lower portion of the colon by sigmoidoscopy or full endoscopic visualization by colonoscopy; the sensitivity for detecting cancer is 15-30%, 60% and 90%, respectively. Early detection and treatment is critical to a favorable treatment outcome. See Biology and Treatment of Colorectal Cancer Metastasis (1986) A. M. Mastormarino ed., Martinus Nijhoff, and Molecular Genetics and Colorectal Neoplasia: A Primer for the Clinician (1996) Church et al. eds. Igaku-Shoin Medical Pub., which are each incorporated herein by reference.

Colorectal cancer stages may be classified according to the Dukes' system, which reflects how deeply the cancer has invaded the lining or wall of the bowel, and whether it has spread to the lymph nodes or more distant sites. See Table 1. The Dukes' stages are as follows: Dukes' A, B, C and D. Dukes' A is characterized by superficial invasion into the mucosa, the innermost muscular layer of the bowel wall (i.e., nearest the stool). Those with cancers detected at Dukes' A stage have a greater than 90% five-year survival rate. Dukes' B, which can be further divided into B1 and B2, is characterized by penetration of the cancer into or through the muscular layer of the bowel wall but not into the regional lymph nodes. Dukes' C, which can be further divided into C1 and C2, is characterized by spread of the cancer to regional lymph nodes. Dukes' D is characterized by metastasis of the cancer to distant organs such as the liver. The five-year survival rate for individuals with Dukes' D is less than 1%. The risk of recurrence of the cancer or metastasis to other parts of the body increases from Dukes' A to Dukes' C stages.

TABLE 1
Dukes' Classification of Colon Cancer

| | EXTENT OF | LYMPH NODE | |
|---|---|---|---|
| CLASS | INVASION | INVOLVEMENT | PROGNOSIS |
| Dukes' A | Limited to the | None | 5 year survival > 90% |
| | mucosa | | |
| Dukes' B1 | into muscularis | None | 5 year survival 70-85% |
| | propria | | |
| Dukes' B2 | through | None | 5 year survival 55-65% |
| | muscularis | | |
| | propria | | |
| Dukes' C1 | into muscularis | Yes | 5 year survival 45-55% |
| | propria | | |
| Dukes' C2 | through | Yes | 5 year survival 20-30% |
| | muscularis | | |
| | propria | | |
| Dukes' D | distant | NA | 5 year survival < 1% |
| | metastases | | |

Surgical resection is highly effective for early stage colon cancers, providing cure rates of over 90% in Dukes' A and 75% in Dukes' B. The presence of nodal involvement (Dukes' C) predicts a 60% likelihood of recurrence. An important factor in colorectal cancer prognosis is the presence or absence of regional lymph node metastasis. In one embodiment mRNA expression profiles associated with the presence or absence of nodal involvement are disclosed. In some embodiments molecular profiling of primary tumors may be used to distinguish lymph node negative and positive stages of colorectal cancers. In one embodiment a molecular signature of lymph node metastasis is disclosed. In some embodiments a molecular signature is used, for example, to determine disease stage (e.g. presence or absence of lymph node metastasis), predict treatment outcome, select treatment options (e.g. radiation and chemotherapy), identify new therapeutic targets or identify compounds that promote or inhibit metastasis or disease progression.

Methods are disclosed for predicting phenotypic classes of colorectal tumors. Methods are also disclosed for the identification of compounds that modulate the transition between a Dukes' B and Dukes' C tumor or compounds that may modulate metastasis of a colorectal tumor into the lymph nodes, based on gene expression profiles. In one aspect, the method involves identifying a colorectal tumor by obtaining a nucleic acid sample derived from colorectal tissue and determining a gene expression profile from a gene expression product of at least one informative gene having altered expression in a Dukes' B type tumor compared to a Dukes' C type tumor. In a preferred embodiment the expression of a plurality of genes is analyzed and compared to the expression of the same genes in reference samples. For some informative genes expression is decreased in Dukes' C tumors relative to Dukes' B tumors and decreased expression in the unknown sample compared to expression levels in Dukes' B tumors is indicative of a Dukes' C tumor. For some informative genes expression is increased in Dukes' C tumors relative to Dukes' B tumors and increased expression in the unknown sample compared to expression levels in Dukes' B tumors is indicative of a Dukes' C tumor.

Comparison of gene expression patterns from Dukes' B and Dukes' C samples resulted in the identification of genes that are differentially regulated between the two tumor stages. The genes are listed in Tables 2 and 3. Dukes' C tumors are characterized by nodal involvement indicating metastasis to the lymph nodes. Dukes' B tumors are characterized by penetration of the tumor into the wall of the bowel but not into the surrounding lymph nodes. Different treatment regimens are indicated by the stage of the tumor. Treatments available include, but are not limited to, surgical resection, chemotherapy and radiation treatment. The genes that are differentially regulated in Dukes' C versus Dukes' B tumors may be used as a predictive signature of metastases or of regional lymph node involvement.

Differential gene expression has been used to differentiate benign colorectal tumors from malignant tumors (Notterman et al., (2001). Cancer Res 61(7):3124-30), to distinguish colon carcinomas from normal samples (Zou, T. T. and Meltzer, S. J. (2002) Oncogene 21931):4855-62), to identify genes that are differentially expressed in highly metastatic cell lines (Hegde, P. and Quakenbush, J. (2001) Cancer Res 61:7792-7797), and to identify genes whose expression was different between adenomas and carcinomas (Lin, Y. M. and Nakamura, Y. (2002) Oncogene 21(26):4120-8), each of which is incorporated herein by reference.

In one embodiment one or more of the genes identified in Tables 2-3 may be used to characterize a sample as either having the presence or absence of regional lymph node metastases. The expression level of one or more of the genes listed in Tables 2-3 is determined by any method known in the art and the expression level is compared to the expression level of the same one or more genes from a second source that is known or predicted to be free of regional lymph node metastases. The second source may be, for example, a tissue sample that has been classified to be free of regional lymph node metastases, such as a Dukes' B tumor sample, or an average expression value from two or more samples that are believed to be free of regional lymph node metastases. The direction and magnitude of the relative change in the expression value in the unknown sample compared to the reference sample may then be compared to a database of expression changes between samples that have regional lymph node metastases present and samples where regional lymph node metastases is absent. If a gene is up regulated in an unknown sample when compared to a non metastasized sample and that gene is up regulated in a metastasized sample, such as Dukes' C, compared to a non metastasized sample, such as a Dukes B' sample, for example, as shown in Tables 2-3, then the sample is classified as having regional lymph node metastases present. For example, if gene A is up regulated in the unknown sample relative to the average expression of gene A in a plurality of Dukes' B samples and gene A is up regulated in Dukes' C samples relative to Dukes' B samples then the unknown sample is classified as being Dukes' C and having regional lymph node metastases present. In a preferred embodiment a collection of genes is evaluated so that classifications may be made with high confidence. In a more preferred embodiment at the expression level of at least 20 genes from Tables 2 and 3 are compared to a reference.

In some embodiments a plurality of informative genes will be analyzed. Each informative gene may be up or down regulated in the unknown sample compared to a non-metastasized sample. The direction of change for each informative gene is compared to a database of direction of change for informative genes. The database may be generated by comparing gene expression in one or more samples where metastasis is present to one or more samples where metastasis is absent, such as the comparison of Dukes' C tumors to Dukes' B tumors in Tables 2 and 3. If the direction of change between the unknown sample and a non-metastasized sample or samples is the same as the direction of change between the metastasized and non-metastasized samples in a database then the unknown sample is predicted to have regional lymph node metastasis present. For example, Table 2 indicates that Spondin 1 (SEQ ID NO: 4) is up regulated in Dukes' C samples compared to Dukes' B samples so if expression levels of Spondin 1 are determined in an unknown sample and found to be up regulated in comparison to the expression of Spondin 1 in one or more Dukes' B samples, or the average expression level of Spondin 1 in a plurality of Dukes' B samples, then the unknown sample is predicted to have metastasized. If many genes from the set of informative genes are analyzed the probability that the prediction is correct increases. Class prediction with the 81 genes disclosed in Tables 2 and 3 (SEQ ID NOs: 1-81) resulted in classification with greater than 90% accuracy. Each of the 81 genes identified is represented in Tables 2 and 3. The sequence of the gene may be obtained by the GenBank accession number and the sequences of the probes used to detect the gene may be obtained from the Affymetrix NetAffx.com website.

In some embodiments a collection of genes that are differentially expressed between Dukes' B and Dukes' C stages is disclosed. A collection of 81 genes were identified and are disclosed in Tables 2 and 3, SEQ ID NOs: 1-81. The collection may comprise each of the 81 genes or a subset of the 81 genes. A training data set may be provided that includes expression level values for each of the genes in the collection in a plurality of reference samples of known Dukes' stage or known to have presence or absence of lymph node metastasis. Expression values may be obtained by any method known in the art. The training data set may include the average expression value for a given gene in a plurality of different reference samples that are of similar phenotypic class (e.g. the average expression value for H2BFH in a plurality of samples that are each Dukes' stage B). Individual genes in the unknown sample are compared to the corresponding gene in the reference sample or samples, for example, the expression of H2BFH in the unknown sample is compared to the expression of H2BFH in the reference sample or samples.

In one embodiment an array of nucleic acid probes is designed to interrogate one or more genes from Tables 2 and 3. There are 81 unique genes represented in the tables. In a preferred embodiment an array is designed to interrogate each of the 81 genes or a subset of the 81 genes. In a preferred embodiment the array may contain a limited number of probes designed to analyze only a specific set of genes that are part of a gene expression profile, for example in one embodiment an array is disclosed that has a probe set for each of the genes in Tables 2 and 3 and control probes. Control probes that may be used include the controls currently described in the Affymetrix, GeneChip® Expression Analysis Technical Manual in Chapter 2, Section 2, for example, Control Oligo B2, biotinylated hybridization controls: bioB, bioC, bioD and cre, and the Poly-A spike controls: dap, thr, trp, phe and lys. The arrays may be used to identify changes in gene expression pattern in the target genes between two samples or to obtain an expression pattern from an experimental sample. The expression pattern may be compared to expression patterns of known reference samples. Samples may be differentially labeled and hybridized to the same copy of an array. Alternatively, samples may be hybridized to different copies of an array.

The methods may be used as a predictor of likelihood of the presence or absence of regional lymph node metastases. The methods may be combined with other methods of classification, such as histological methods, to provide a determination of the presence or absence of regional lymph node metastases or to increase the confidence level of a classification based on a second method.

In one embodiment the genes identified may be used individually or in groups of 2 or more, 3 or more, 4 or more, 5 or more, 6 or more, 10 or more or 20 or more to determine the efficacy of a drug or treatment regimen. In preferred embodiments each of the 81 genes is analyzed. For example, tumor cells from a Dukes' C tumor may be treated with a drug and the expression level of one or more informative genes may be determined before and after treatment to determine the direction of change in expression. If the direction of change is the same as the direction of change from Dukes' C to Dukes' B tumors then the drug is a possible inhibitor of metastasis.

In another embodiment the genes may be used individually or in groups of 2 or more, 3 or more, 4 or more, 5 or more, 6 or more, 10 or more or 20 or more to identify compounds or environmental stimuli that may increase or decrease the likelihood of metastasis. For example, tumor cells from a Dukes' B tumor may be treated with a compound and the expression level of one or more informative genes from Tables 2-3 may be determined before and after treatment to determine the direction of change. If the direction of change is the same as the direction of change between Dukes' B and Dukes' C tumors then the compound increases the likelihood of metastasis. If the direction of change is different or the opposite of the direction of change between Dukes' B and Dukes' C tumors, for example if a gene is up regulated in Dukes' C compared to Dukes' B and treatment with the compound down regulates the expression of the gene, then the compound may be an inhibitor of metastasis.

Chemotherapy and radiation normally result in adverse side effects. These treatments have been shown to reduce the risk of recurrence and to increase cure rate but not everyone who has the treatment will benefit and some patients may benefit from one treatment but not the other. Currently there is no way of telling in advance who will or will not be helped by a given treatment. In some embodiments the gene expression patterns disclosed may be used to predict efficacy of a given treatment or to predict treatment outcome. Patients who will respond to a particular treatment may be identified using gene expression pattern.

Hierarchical clustering analysis was applied to assess the distinction between Dukes' B and Dukes' C samples based on their expression profiles. In two-dimensional analyses, gene expression patterns that are similar, group together, i.e., cluster. Consequently, one expects that tissues with markedly different expression patterns would form distinct clusters when sorted by expression pattern. Results of this analysis show good segregation of Dukes' B and Dukes' C tissues based on the differing levels of the candidate genes. Clustering with the genes gave clear separation between Dukes' B and Dukes' C type tumors. In some embodiments any 2 or more of these genes may be used as a predictive signature of metastasis.

Tables 2 and 3 disclose a collection of genes that were identified as being differentially expressed in Dukes' B and Dukes' C samples. Some genes were identified as being up regulated in the Dukes' C samples compared to the Dukes' B samples and other genes were identified as being down regulated in the Dukes' C samples compared to the Dukes' B samples. Direction and magnitude of change is indicated in column 8, for example H2BFH is up-regulated in C relative to B by 1.5 fold and FUT4 is down regulated in C relative to B by 1.5 fold. Column 1 indicates the SEQ ID NO of the gene, there are 20 genes that are present in both Table 2 and Table 3 for a total of 81 genes. Column 2 is a numerical identifier of the probe set for the gene on the Affymetrix HU133 probe array (Affymetrix, Inc., Santa Clara, Calif.) the sequences of the probes in the corresponding probe set and the target sequence used to generate the probes can be accessed on the web at netaffx.com. Column 3 lists the GenBank accession number (using the accession number the entire gene and surrounding sequence may be accessed on the world wide web at ncbi.nlm.nih.gov/Entrez/index.html). Column 4 includes a description of the gene. Column 5 provides the gene symbol if known. Column 6 provides the chromosomal location of the gene. Column 6 provides the Mann-Whitney U test P-value, Column 7 indicates the fold change of the expression of the gene in Dukes' C relative to B. The direction of change is indicated in Column 8, for example, H2BFH is up regulated in Dukes' C relative to B by 1.6 fold, meaning that the expression of H2BFH in the Dukes' C samples is 1.6X the expression of H2BFH in the Dukes' B samples and the expression of FUT4 is down regulated in Dukes' C relative to B by 1.5 fold, indicating that the expression of FUT4 in the Dukes' B samples is 1.5X the expression of FUT4 in the Dukes' C samples. Column 9 indicates if the gene was up or down regulated in Dukes' C relative to Dukes' B. Column 10 indicates the functional category of the gene if known.

In one embodiment gene expression profiles are used to distinguish different Dukes' stages of colon tumors. In another embodiment gene expression profiles are used to determine if a tumor has metastasized to the local lymph nodes. In one embodiment samples are hybridized to an array to detect differential gene expression. In another embodiment quantitative RT-PCR is used to detect gene expression differences or to confirm gene expression differences observed by another method. In one embodiment hierarchical clustering using genes differentially expressed between different Dukes' stages of tumors, for example Dukes' B versus Dukes' C, is used to segregate tissue types. Hierarchical clustering of genes may be used to increase confidence in genes that are candidates for a molecular signature of a particular disease state. In another embodiment candidate genes that have been identified are validated using a larger patient cohort. In another embodiment the differentially expressed genes disclosed are used to develop new or improved diagnostics methods and new treatment regimens. In another embodiment one or more of the genes in Tables 2 or 3 are used as candidates for drugs to regulate, inhibit or prevent transition from Dukes' B stage to Dukes' C stage. In another embodiment one or more of the genes in Tables 2 or 3 are candidates for genes that play a role in metastasis.

The above disclosure generally describes the present invention. A more complete understanding can be obtained by reference to the following specific examples which are provided herein for purposes of illustration only, and are not intended to limit the scope of the invention.

### EXAMPLE

Molecular profiling was carried out by using 20 samples collected from colon cancer patient-donors without known local lymph node metastases (Dukes' Stage B) and 15 samples from patient-donors with local lymph node metastases (Dukes' Stage C). The maximum age of the patients with Dukes' B was 96 years, minimum 46 years, average 74 years STD=12.5 years. The maximum age of the patients with Dukes' C was 86 years, minimum 36 years, average 67.6 years STD=14 years. Labeled target cRNAs were hybridized to high density oligonucleotide arrays containing sequences of approximately 22,000 genes. The reproducibility of the GeneChip®Probe Array-based platform was tested first by conducting independent sample preparation and hybridizations of one Dukes' C and two Dukes' B samples in triplicate. Reproducibility was evaluated by using various parameters, such as %CV and R squared values. For sample prep methods, hybridization methods and data analysis methods see Affymetrix Gene Expression Technical Manual, 2002, available at Affymetrix.com.

Differentially expressed genes between Dukes' B and Dukes' C patients were identified by the non-parametric, Mann-Whitney U test, the parametric Student t-test and by SAM (Significance Analysis of Microarray, see Tusher et al, (2001) Proc. Natl. Acad. Sci., 98:5116, which is incorporated herein by reference in its entirety) with p ≦0.005 or with p ≦0.01 and a minimum 1.5 fold change between groups. 50 candidate probe sets were identified with P ≦0.005 (Table 3) and 54 probe sets were identified with P ≦0.01 and ≧1.5 fold change (Table 2). After hierarchical clustering, the candidate probe sets accurately segregated Dukes' B from Dukes' C patients. Probe sets were annotated with locus and functional category information. There were 20 probe sets that were detected by both criteria and are present in both Table 2 and Table 3. SEQ ID NOs: 102 to 321 are the probes in the 20 common probe sets, and SEQ ID NOs: 82-101 are the target sequence used to select probes for these probe sets. The probe sets are as follows: 214238_AT (SEQ ID NOs: 102-112), 215534_AT (SEQ ID NOs: 113-123), 220583_AT (SEQ ID NOs: 124-134), 207451_AT (SEQ ID NOs: 135-145), 202320_AT (SEQ ID NOs: 146-156),215019_x_AT (SEQ ID NOs: 157-167),211265_AT (SEQ ID NOs: 168-178), 209791_AT (SEQ ID NOs: 179-189), 219103_AT (SEQ ID NOs: 190-200), 201053_S_AT (SEQ ID NOs: 201-211), 220144_S_AT (SEQ ID NOs: 212-222), 212650_AT (SEQ ID NOs: 223-233), 200906_S_AT (SEQ ID NOs: 234-244), 203311_S_AT (SEQ ID NOs: 245-255), 205450_AT (SEQ ID NOs: 256-266), 208546_X_AT (SEQ ID NOs: 267-277), 208920_AT (SEQ ID NOs: 278-288), 210536_S_AT (SEQ ID NOs: 289-299), 210551_S_AT (SEQ ID NOs: 300-310), and 212253_X_AT (SEQ ID NOs: 311-321). For example, probe set “214238_at” measures expression of the DT1P1B gene (SEQ ID NO: 52), the probe set includes the eleven perfect match probes SEQ ID NO: 102 to 112. The probe set also includes mismatch probes that vary from the perfect match probe at the central position, position 13 in a 25 nucleotide probe. When 3′ amplification methods are used probes are typically selected from the terminal 600 bases of the mRNA. When antisense cRNA will be hybridized to the array, the probes are complementary to the antisense cRNA and are therefore the same orientation and sequence as the sense MRNA.

The chromosomal distribution of 48 of the identified genes differentially expressed in Duke B vs. Duke C patients, detected by both Mann-Whitney U test and Student T test as well as SAM with P<=0.005 and False Detection Rate (FDR)=0, is as follows. For down regulated genes (chromosome/number of genes identified): 1/3, 2/1, 3/0, 4/0, 5/1, 6/0, 7/2, 8/0, 9/0, 10/1, 11/1, 12/1, 13/0, 14/0, 15/0, 16/0, 17/1, 18/0, 19/1, 20/4, 21/1, 22/1, X/1, and Y/1. For up regulated genes 1/3, 2/2, 3/0, 4/1, 5/1, 6/1, 7/2, 8/1, 9/0, 10/0, 11/2, 12/2, 13/0, 14/6, 15/1, 16/1, 17/1, 18/0, 19/2, 20/0, 21/0, 22/0, X/1, and Y/0. More than 20% of the candidates overexpressed by Dukes' C are located on Chromosome 14, while about 20% of the candidates underexpressed by Dukes' C. are located on Chromosome 20, which indicates possible hyper or hypomethylation on selected chromosomes.

Many candidate genes were found to involve important pathways. Candidate gene RAS is involved in metastasis. FZ (frizzled) and DSH (disheveled), members of the Wnt signaling pathway, were inversely correlated in their expression.

Quantitative-RT- PCR (QRT-PCR) of selected candidate genes confirmed the microarray results for all candidates tested. QRT-PCR may be done by removing DNA from total RNA by on-column DNase digestion (Qiagen). Primers and probe selection may be done using PrimerExpress (ABI). Reagents for RT and PCR may be obtained from ABI. Q-RT-PCR may be done using Taqman and an ABI 7700. SequenceDetector Analysis Software 1.7 is available from ABI. QRT-PCR validation of microarray results was performed for 10 of the candidate genes. Dukes' C/Dukes' B fold change were compared by QRT-PCR and microarray data for 10 candidate genes with P<=0.01 and fold change >=1.5 in microarray detection. The 5 up-regulated genes analyzed were synaptogyrin 3 (SYNGR 3), growth factor receptor-bound protein 14(GRB14), RAS-related protein 7 (RAB7), spondin1(SPON1), dishevelled 2 (DVL2). The 5 down-regulated genes analyzed were translation initiation factor 1A (EIF1A), proliferating cell nuclear antigen (PCNA), proteasome inhibitor subunit 1 (PSMF1), frizzled homolog 3 (FZD3) and apoptosis-related cysteine protease (CASP5). EIF1A, SPON1 and PCNA have been previously identified. RAB7 is known to be involved in the metastasis pathway and DVL2 and FZD3 are known to be involved in the Wnt signaling pathway.

cDNA Synthesis was done using SuperScript Choice (Invitrogen) system with primer: T7-dT with sequence comprising a promoter for T7 RNA polymerase in the 5′ region and (dT)24 at the 3′ end. In Vitro Transcription was done using BioArray High Yield RNA Transcript labeling kit (Enzo). GeneChip® Arrays Human Genome U133A Array (Affymetrix) containing probe sets representing ˜22000 genes were used for hybridization. For Data Analysis the Microarray Suite 5.0 using default parameter settings (Affymetrix), Data Mining Tool (Affymetrix), MS Excel and Access, GeneMaths (Applied Maths) was used. See also, Mahadevappa and Warrington, (1999), Nature Biotech. 17:1134 which is incorporated herein by reference in its entirety. In preferred embodiments sample preparation and array analysis are performed according to the methods described in the GeneChip® Expression Analysis Technical Manual, rev 3 (2003, 2004) available from Affymetrix, Inc., Santa Clara, which is incorporated herein by reference in its entirety for all purposes.

Mann-Whitney U test may be carried out on raw data after removing probe sets called absent in all samples in the study and selecting candidate genes with a selected P value. RNA integrity of the samples may be evaluated using GAPDH ratio and percent present calls.

The example shows that gene expression profiling can accurately segregate invasive colon cancers with and without lymph node metastases. Hierarchical clustering as well as 3D PCA of 50 probe sets commonly detected by U test and T test with P<=0.005 (Table 3) or 54 probe sets with P<=0.01 and average fold Dukes' C versus Dukes' B >=1.5 (Table 2) separate all the samples in Dukes' B from Dukes' C with a high degree of accuracy. Q-RTPCR results from 10 candidate genes were consistent with the observed microarray results. Class prediction with 81 unique probe sets (50+54) may be used to classify Duke's B and Duke's C with a high level of accuracy, greater than 90% accuracy, through 10 fold cross validation. Three of the candidate genes, EIF1A, SPON1 and PCNA, had been previously identified in other studies, confirming the accuracy of the results.

### CONCLUSION

It is to be understood that the above description is intended to be illustrative and not restrictive. Many variations of the invention will be apparent to those of skill in the art upon reviewing the above description. The scope of the invention should be determined with reference to the appended claims, along with the full scope of equivalents to which such claims are entitled. All cited references, including patent and non-patent literature, are incorporated herewith by reference in their entireties for all purposes.

TABLE 2
Genes differentially expressed in Dukes' C relative to Dukes B with P <= 0.01 and fold change >=1.5

| | | | | | | | | Change | |
|---|---|---|---|---|---|---|---|---|---|
| SEQ ID | Probeset | | | | Chromosome | | Fold | Direction | Functional |
| NO | Name | Accession | Description | Symbol | Loci | P-Value | Change | C to B | Category |
| 1 | 208527_x_at | NM_003523 | H2B histone | H2BFH | 6p21.3 | 0.0057 | 1.6 | up | chromosome |
| | | | family | | | | | | |
| 2 | 208546_x_at | NM_003524 | H2B histone | H2BFJ | 6p21.3 | 0.00459 | 2.1 | up | chromosome |
| | | | family, member J | | | | | | |
| | | | (H2BFJ) | | | | | | |
| 3 | 221115_s_at | NM_018655 | lens epithelial | LENEP | 1q22 | 0.00783 | 1.6 | up | developmental |
| | | | protein | | | | | | |
| 4 | 213994_s_at | AI885290 | spondin 1, (f- | SPON1 | 11p14-p15.2 | 0.00459 | 2.1 | up | extracellular |
| | | | spondin) | | | | | | |
| | | | extracellular | | | | | | |
| | | | matrix protein | | | | | | |
| 5 | 209892_at | AF305083 | alpha(1,3)- | FUT4 | 11q21 | 0.00634 | 1.5 | down | metablism |
| | | | fucosyltransferase | | | | | | |
| | | | IV (FUTIV) | | | | | | |
| 6 | 204476_s_at | NM_022172 | pyruvate | PC | 11q13.4-q13.5 | 0.00868 | 1.6 | up | metabolism |
| | | | carboxylase (PC) | | | | | | |
| 7 | 211407_at | M33374 | cell adhesion | NDUFB7 | 19p13.12-p13.11 | 0.00705 | 1.9 | down | metabolism |
| | | | protein (SQM1) | | | | | | |
| 8 | 209791_at | AL049569 | PDI (protein- | PADI2 | 1p35.2-p35.1 | 0.001 | 1.9 | up | metabolism |
| | | | arginine | | | | | | |
| | | | deiminase | | | | | | |
| 9 | 210536_s_at | S67798 | PH-20 (SPAM1) | SPAM1 | 7q31 | 0.00459 | 1.8 | up | metabolism |
| 10 | 205450_at | NM_002637 | phosphorylase | PHKA1 | Xq12-q13 | 0.00233 | 1.5 | up | metabolism |
| | | | kinase, alpha 1 | | | | | | |
| | | | (muscle) (PHKA1) | | | | | | |
| 11 | 207451_at | NM_014360 | phosphorylase | NKX2H | 14q13.1 | 0.00207 | 1.7 | up | oncogenesis |
| | | | kinase, alpha 1 | | | | | | |
| | | | (muscle) (PHKA1) | | | | | | |
| 12 | 217268_at | AK024417 | RAS-RELATED | RAB7 | 3q22.1 | 0.00961 | 2.1 | up | oncogenesis |
| | | | PROTEIN RAB-7 | | | | | | |
| 13 | 210551_s_at | BC001620 | acetylserotonin O- | ASMT | Xp22.3 or | 0.00207 | 2.1 | down | protein |
| | | | methyltransferase | | Yp11.3 | | | | synthesis |
| 14 | 207500_at | NM_004347 | apoptosis-related | CASP5 | 11q22.2-q22.3 | 0.0057 | 1.9 | down | proteolysis |
| | | | cysteine protease | | | | | | |
| | | | (CASP5) | | | | | | |
| 15 | 201053_s_at | NM_006814 | proteasome | PSMF1 | 20p12.2-p13 | 0.00114 | 1.6 | down | proteolysis |
| | | | (prosome, | | | | | | |
| | | | macropain) | | | | | | |
| | | | inhibitor subunit 1 | | | | | | |
| | | | (PI31) (PSMF1) | | | | | | |
| 16 | 216981_x_at | X60502 | leukosialin cDNA- | SPN | 16p11.2 | 0.00868 | 1.5 | up | receptor/signal |
| | | | II | | | | | | transduction |
| 17 | 211265_at | U13216 | protaglandin | PTGER3 | 1p31.2 | 0.00368 | 1.7 | down | receptor/signal |
| | | | receptor EP3A1 | | | | | | transduction |
| 18 | 216247_at | AF113008 | ribosomal protein | FLB0708 | 8 | 0.00634 | 1.6 | down | ribosomal |
| | | | S20 | | | | | | |
| 19 | 216553_x_at | AL121890 | 40S ribosomal | RPS21 | 20 | 0.00512 | 1.9 | down | ribosomal |
| | | | protein S21 | | | | | | |
| 20 | 204874_x_at | NM_003933 | BAI1-associated | BAIAP3 | 16p13.3 | 0.00459 | 2.0 | up | signal |
| | | | protein 3 | | | | | | transduction |
| | | | (BAIAP3) | | | | | | |
| 21 | 214067_at | AL031709 | C2 domain | KIAA0734 | 16p13.3 | 0.0057 | 1.6 | down | signal |
| | | | protein KIAA0734 | | | | | | transduction |
| 22 | 218759_at | NM_004422 | dishevelled 2 | DVL2 | 17p13.2 | 0.00705 | 1.8 | up | signal |
| | | | (homologous to | | | | | | transduction |
| | | | Drosophila dsh) | | | | | | |
| | | | (DVL2) | | | | | | |
| 23 | 206204_at | NM_004490 | growth factor | GRB14 | 2q22-q24 | 0.0057 | 2.6 | up | signal |
| | | | receptor-bound | | | | | | transduction |
| | | | protein 14 | | | | | | |
| | | | (GRB14) | | | | | | |
| 24 | 219683_at | NM_017412 | frizzled | FZD3 | 8p21 | 0.00783 | 1.8 | down | signal |
| | | | (Drosophila) | | | | | | transduction |
| | | | homolog 3 (FZD3) | | | | | | |
| 25 | 215053_at | AK023808 | transcriptional | SRCAP | 16p11.1 | 0.00961 | 1.5 | up | transcription |
| | | | activator SRCAP | | | | | | |
| | | | (SRCAP) | | | | | | |
| 26 | 202320_at | NM_001520 | general | GTF3C1 | 16p12 | 0.00294 | 1.5 | up | transcription |
| | | | transcription | | | | | | |
| | | | factor IIIC, | | | | | | |
| | | | polypeptide 1 | | | | | | |
| | | | (alpha subunit, | | | | | | |
| | | | 220 kD) | | | | | | |
| | | | (GTF3C1) | | | | | | |
| 27 | 211182_x_at | AF312387 | AML1AMP19 | RUNX1 | 21q22.3 | 0.00262 | 1.8 | up | transcription |
| | | | fusion protein | | | | | | |
| | | | (AML1AMP19 | | | | | | |
| | | | fusion) | | | | | | |
| 28 | 201018_at | AL079283 | eukaryotic | EIF1A | X | 0.00868 | 1.5 | down | transcription |
| | | | translation | | | | | | |
| | | | initiation factor 1A | | | | | | |
| 29 | 203961_at | AL157398 | nebulette protein | NEBL | 10p12 | 0.00233 | 1.6 | down | structure |
| | | | (NEBL, actin- | | | | | | |
| | | | binding Z-disc | | | | | | |
| | | | protein) | | | | | | |
| 30 | 205691_at | NM_004209 | synaptogyrin 3 | SYNGR3 | 16p13 | 0.00961 | 3.5 | up | structure |
| | | | (SYNGR3) | | | | | | |
| 31 | 203407_at | NM_002705 | periplakin (PPL) | PPL | 16p13.3 | 0.0057 | 1.5 | up | structure |
| 32 | 214870_x_at | AC002045 | nuclear pore | NPIP | 16p13-p11 | 0.00705 | 1.8 | up | structure |
| | | | complex | | | | | | |
| | | | interacting protein | | | | | | |
| 33 | 212253_x_at | BG253119 | KIAA0728 protein | BPAG1 | 6p12-p11 | 0.00329 | 1.7 | up | structure |
| 34 | 208920_at | AV752215 | sorcin | SRI | 7q21.1 | 0.00368 | 1.8 | down | structure |
| 35 | 210454_s_at | U24660 | G protein | KCNJ6 | 21q22.13-q22.2 | 0.00411 | 2.0 | down | transport |
| | | | coupled inward | | | | | | |
| | | | rectifier potassium | | | | | | |
| | | | channel 2 | | | | | | |
| | | | (hiGIRK2) | | | | | | |
| 36 | 203311_s_at | M57763 | ADP-ribosylation | ARF6 | 7q22.1 | 0.000886 | 1.5 | up | transport |
| | | | factor (hARF6) | | | | | | |
| 37 | 218224_at | NM_006029 | paraneoplastic | PNMA1 | 14q24.1 | 0.00961 | 1.6 | up | tumor related |
| | | | antigen MA1 | | | | | | |
| | | | (PNMA1) | | | | | | |
| 38 | 219103_at | NM_017707 | hypothetical | UPLC1 | 1p36.11 | 0.00368 | 1.9 | up | tumor related |
| | | | protein FLJ20199 | | | | | | |
| 39 | 211568_at | AB011122 | brain-specific | BAI3 | 6q12 | 0.00294 | 1.6 | up | tumor related |
| | | | angiogenesis | | | | | | |
| | | | inhibitor 3 | | | | | | |
| 40 | 216462_at | X79200 | SYT-SSX protein | SSX2 | Xp11.23-p11.22 | 0.00783 | 2.2 | down | tumor related |
| 41 | 220583_at | NM_025086 | hypothetical | FLJ22596 | 11q13.3 | 0.00164 | 2.0 | up | unknown |
| | | | protein FLJ22596 | | | | | | |
| 42 | 220278_at | NM_018039 | FLJ10251 | FLJ10251 | 11q21 | 0.00783 | 2.2 | up | unknown |
| 43 | 222170_at | AF098968 | familial | AF098968 | 16p13.3 | 0.00868 | 2.1 | down | unknown |
| | | | Mediterranean | | | | | | |
| | | | fever locus region | | | | | | |
| 44 | 215019_x_at | AW474158 | weakly similar to | KIAA1827 | 19q13 | 0.00294 | 2.1 | up | unknown |
| | | | ZINC FINGER | | | | | | |
| | | | PROTEIN 83 | | | | | | |
| 45 | 212358_at | AL117468 | cDNA | CLIPR-59 | 19q13.13 | 0.00411 | 1.6 | up | unknown |
| | | | DKFZp586N1922 | | | | | | |
| 46 | 220144_s_at | NM_022096 | hypothetical | ANKRD5 | 20pter-q11.23 | 0.00207 | 1.9 | down | unknown |
| | | | protein FLJ21669 | | | | | | |
| 47 | 212650_at | BF116032 | KIAA0903 protein | KIAA0903 | 2p13.3 | 0.00207 | 1.6 | up | unknown |
| 48 | 200906_s_at | AK025843 | palladin | KIAA0992 | 4q32.3 | 0.00233 | 1.5 | up | unknown |
| 49 | 221145_at | NM_018499 | PRO1097 | FLB4237 | 8q22.1 | 0.00783 | 2.1 | up | unknown |
| | | | (PRO1097) | | | | | | |
| 50 | 212444_at | AA156240 | clone HRC00953 | clone HRC00953 | 12 | 0.00868 | 1.6 | up | unknown |
| 51 | 213411_at | AW242701 | cDNA | cDNA | 7 | 0.00329 | 1.5 | down | unknown |
| | | | DKFZp434E0528 | DKFZp434E0528 | | | | | |
| 52 | 214238_at | AI093572 | clone DT1P1B6 | clone DT1P1B6 | 2 | 0.00262 | 1.5 | down | unknown |
| 53 | 215534_at | AL117546 | cDNA | cDNA | 5 | 0.00262 | 2.5 | up | unknown |
| | | | DKFZp586C1923 | DKFZp586C1923 | | | | | |
| 54 | 216144_at | AL137378 | cDNA | cDNA | 7 | 0.0057 | 1.5 | up | unknown |
| | | | DKFZp434K1126 | DKFZp434K1126 | | | | | |

TABLE 3

| SEQ | | | | | | | | Change | |
|---|---|---|---|---|---|---|---|---|---|
| ID | Probeset | | | | Chromosome | | Fold | Direction | Functional |
| NO | Name | Accession | Description | Symbol | Loci | P-Value | Change | C to B | Category |
| 11 | 207451_at | NM_014360 | NK-2 homolog H | NKX2H | 14q13.1 | 0.00207 | 1.7 | up | oncogenesis/ |
| | | | (Drosophila) | | | | | | transcription |
| 55 | 213889_at | AI742901 | phosphatidylinositol | PIGL | 17p12-p11.2 | 0.00368 | 1.4 | down | biosynthesis |
| | | | glycan, class L | | | | | | |
| 56 | 210624_s_at | BC0000109 | ilvB (bacterial | ILVBL | 19p13.1 | 0.000886 | 1.3 | down | biosynthesis |
| | | | acetolactate | | | | | | |
| | | | synthase)-like | | | | | | |
| 57 | 211928_at | AB002323 | dynein, | DNCH1 | 14q32.3-qter | 0.000886 | 1.5 | up | cell cycle |
| | | | cytoplasmic, | | | | | | |
| | | | heavy | | | | | | |
| | | | polypeptide 1 | | | | | | |
| 2 | 208546_x_at | NM_003524 | H2B histone | H2BFJ | 6p21.3 | 0.00459 | 2.1 | up | Chromosome |
| | | | family, member J | | | | | | |
| 58 | 40489_at | Cluster Incl | dentatorubral- | DRPLA | 12p13.31 | 0.00114 | 1.4 | up | development |
| | | D31840 | pallidoluysian | | | | | | |
| | | | atrophy (atrophin- | | | | | | |
| | | | 1) | | | | | | |
| 59 | 212751_at | BG290646 | ubiquitin- | UBE2N | 12q21.33 | 0.00329 | 1.3 | down | DNA repair |
| | | | conjugating | | | | | | |
| | | | enzyme E2N | | | | | | |
| | | | (UBC13 | | | | | | |
| | | | homolog, yeast) | | | | | | |
| 60 | 214086_s_at | AK001980 | ADP- | ADPRTL2 | 14q11.2-q12 | 0.00368 | 1.4 | up | DNA repair |
| | | | ribosyltransferase | | | | | | |
| | | | (NAD+; | | | | | | |
| | | | poly(ADP-ribose) | | | | | | |
| | | | polymerase)-like 2 | | | | | | |
| 8 | 209791_at | AL049569 | peptidyl arginine | PADI2 | 1p35.2-p35.1 | 0.001 | 1.9 | up | Enzyme |
| | | | deiminase, type II | | | | | | |
| 61 | 212407_at | AL049669 | CGI-01 protein | CGI-01 | 1q24-q25.3 | 0.00262 | 1.3 | down | Enzyme |
| 62 | 211969_at | BG420237 | heat shock 90 kD | HSPCA | 14q32.33 | 0.00459 | 1.2 | up | heat shock |
| | | | protein 1α | | | | | | |
| 63 | 218809_at | NM_024960 | pantothenate | PANK2 | 20p13 | 0.00459 | 1.3 | down | kinase |
| | | | kinase 2 | | | | | | |
| | | | (Hallervorden- | | | | | | |
| | | | Spatz syndrome) | | | | | | |
| 64 | 202325_s_at | NM_001685 | ATP synthase, | ATP5J | 21q21.1 | 0.00164 | 1.3 | down | metabolism |
| | | | H+ transporting, | | | | | | |
| | | | mitochondrial F0 | | | | | | |
| | | | complex, subunit | | | | | | |
| | | | F6 | | | | | | |
| 9 | 210536_s_at | S67798 | sperm adhesion | SPAM1 | 7q31 | 0.00459 | 1.8 | up | metabolism |
| | | | molecule 1 (PH- | | | | | | |
| | | | 20 hyaluronidase, | | | | | | |
| | | | zona pellucida | | | | | | |
| | | | binding) | | | | | | |
| 65 | 200641_s_at | U28964 | tyrosine 3- | YWHAZ | 8q23.1 | 0.00262 | 1.3 | up | metabolism |
| | | | monooxygenase/tryptophan | | | | | | |
| | | | 5- | | | | | | |
| | | | monooxygenase | | | | | | |
| | | | activation protein, | | | | | | |
| | | | zeta polypeptide | | | | | | |
| 10 | 205450_at | NM_002637 | phosphorylase | PHKA1 | Xq12-q13 | 0.00233 | 1.5 | up | metabolism |
| | | | kinase, alpha 1 | | | | | | |
| | | | (muscle) | | | | | | |
| 66 | 212159_x_at | AI125280 | adaptor-related | AP2A2 | 11 | 0.000467 | 1.3 | up | others |
| | | | protein complex 2 | | | | | | |
| 44 | 215019_x_at | AW474158 | KIAA1827 protein | KIAA1827 | 19q13 | 0.00294 | 2.1 | up | others |
| 67 | 221549_at | AF337808 | glutamate rich | GRWD | 19q13.33 | 0.000781 | 1.5 | up | others |
| | | | WD repeat | | | | | | |
| | | | protein GRWD | | | | | | |
| 15 | 201053_s_at | NM_006814 | proteasome | PSMF1 | 20p12.2-p13 | 0.00114 | 1.6 | down | proteasome |
| | | | (prosome, | | | | | | |
| | | | macropain) | | | | | | |
| | | | inhibitor subunit 1 | | | | | | |
| | | | (PI31) | | | | | | |
| 13 | 210551_s_at | BC001620 | acetylserotonin | ASMT | Xp22.3 or | 0.00207 | 2.1 | down | protein |
| | | | O- | | Yp11.3 | | | | synthesis |
| | | | methyltransferas | | | | | | |
| 68 | 204741_at | NM_001714 | Bicaudal D | BICD1 | 12p11.2-p11.1 | 0.00459 | 1.5 | up | RNA |
| | | | homolog 1 | | | | | | processing |
| | | | (Drosophila) | | | | | | |
| 36 | 203311_s_at | M57763 | ADP-ribosylation | ARF6 | 7q22.1 | 0.000886 | 1.5 | up | Signal |
| | | | factor 6 | | | | | | transduction |
| 69 | 213795_s_at | AL121905 | protein tyrosine | PTPRA | 20p13 | 0.00184 | 1.4 | down | signal |
| | | | phosphatase, | | | | | | transduction/ |
| | | | receptor type, A | | | | | | receptor |
| 17 | 211265_at | U13216 | prostaglandin E | PTGER3 | 1p31.2 | 0.00368 | 1.7 | down | signal |
| | | | receptor 3 | | | | | | transduction/ |
| | | | (subtype EP3) | | | | | | transcription/ |
| | | | | | | | | | Apoptosis |
| 70 | 202568_s_at | AI745639 | MAP/microtubule | MARK3 | 14q32.3 | 0.00164 | 1.3 | up | structure |
| | | | affinity-regulating | | | | | | |
| | | | kinase 3 | | | | | | |
| 33 | 212253_x_at | BG253119 | bullous | BPAG1 | 6p12-p11 | 0.00329 | 1.7 | up | structure |
| | | | pemphigoid | | | | | | |
| | | | antigen 1, | | | | | | |
| | | | 230/240 kDa | | | | | | |
| 34 | 208920_at | AV752215 | sorcin | SRI | 7q21.1 | 0.00368 | 1.8 | down | structure |
| 71 | 202136_at | BE250417 | adenovirus 5 E1A | BS69 | 10p14 | 0.00294 | 1.1 | down | transcription/ |
| | | | binding protein | | | | | | cell |
| | | | | | | | | | proliferation |
| 26 | 202320_at | NM_001520 | general | GTF3C1 | 16p12 | 0.00294 | 1.5 | up | transcription/ |
| | | | transcription | | | | | | cell |
| | | | factor IIIC, | | | | | | proliferation |
| | | | polypeptide 1 | | | | | | |
| 72 | 201200_at | NM_003851 | cellular repressor | CREG | 1q24 | 0.00128 | 1.4 | down | transcription/ |
| | | | of E1A-stimulated | | | | | | cell |
| | | | genes | | | | | | proliferation |
| 47 | 212650_at | BF116032 | KIAA0903 protein | KIAA0903 | 2p13.3 | 0.00207 | 1.6 | up | tumor related |
| 41 | 220583_at | NM_025086 | hypothetical | FLJ22596 | 11q13.3 | 0.00164 | 2.0 | up | unknown |
| | | | protein FLJ22596 | | | | | | |
| 73 | 219816_s_at | NM_018107 | hypothetical | FLJ10482 | 14q11.1 | 0.000532 | 1.2 | up | unknown |
| | | | protein FLJ10482 | | | | | | |
| 74 | 219670_at | NM_024603 | hypothetical | FLJ11588 | 1p32.3 | 0.00294 | 1.4 | up | unknown |
| | | | protein FLJ11588 | | | | | | |
| 75 | 201581_at | BF572868 | hypothetical | DJ971N18.2 | 20p12 | 0.00459 | 1.5 | down | unknown |
| | | | protein | | | | | | |
| | | | DJ971N18.2 | | | | | | |
| 76 | 204594_s_at | NM_013298 | hypothetical | HSU79252 | 22q13 | 0.000688 | 1.3 | down | unknown |
| | | | protein | | | | | | |
| | | | HSU79252 | | | | | | |
| 77 | 221257_x_at | NM_030793 | hypothetical | SP329 | 5q33.1 | 0.00164 | 1.2 | down | unknown |
| | | | protein SP329 | | | | | | |
| 78 | 217972_at | NM_017812 | hypothetical | FLJ20420 | 7q31.31 | 0.00184 | 1.4 | down | unknown |
| | | | protein FLJ20420 | | | | | | |
| 52 | 214238_at | AI093572 | clone DT1P1B6, | DT1P1B | 2 | 0.00262 | 1.5 | down | unknown |
| | | | CAG repeat | | | | | | |
| | | | region | | | | | | |
| 53 | 215534_at | AL117546 | cDNA | DKFZp586C1923 | 5 | 0.00262 | 2.5 | up | unknown |
| | | | DKFZp586C1923 | | | | | | |
| 79 | 214153_at | BE467941 | clone | ELOVL5 | 6 | 0.00262 | 1.4 | up | unknown |
| | | | IMAGE: 3944293 | | | | | | |
| 80 | 214896_at | AL109671 | cDNA clone | EUROIMAGE | 15 | 0.000532 | 1.6 | up | unknown |
| | | | EUROIMAGE | 29222 | | | | | |
| | | | 29222 | | | | | | |
| 38 | 219103_at | NM_017707 | up-regulated in | UPLC1 | 1p36.11 | 0.00368 | 1.9 | up | unknown |
| | | | liver cancer 1 | | | | | | |
| 46 | 220144_s_at | NM_022096 | ankyrin repeat | ANKRD5 | 20pter-q11.23 | 0.00207 | 1.9 | down | unknown |
| | | | domain 5 | | | | | | |
| 81 | 222154_s_at | AK002064 | DKFZP564A2416 | DKFZP564A2416 | 2q33.1 | 0.00411 | 1.3 | up | unknown |
| | | | protein | | | | | | |
| 48 | 200906_s_at | AK025843 | palladin | KIAA0992 | 4q32.3 | 0.00233 | 1.5 | up | unknown |

we claim:

1. A method of classifying a human colorectal tumor comprising the steps of:
obtaining an unknown sample derived from a human colorectal tumor;
determining the gene expression level of each of at least five informative genes in the unknown sample, wherein informative genes are selected from the group consisting of:
a first group of genes that are expressed at higher levels in Dukes' C tumors than in Dukes' B tumors, wherein the first group consists of H2BFH, H2BFJ, LENEP, SPON1, PC, PADI2, SPAM1, PHKA1, NKX2H, RAB7, SPN, BAIAP3, DVL2, GRB14, SRCAP, GTF3C1, RUNX1, SYNGR3, PPL, NPIP, BPAG1, ARF6, PNMA1, UPLC1, BAI3, FLJ22596, FLJ10251, KIAA1827, CLIPR-59, KIAA0903, KIAA0992, FLB4237, clone HRC00953, cDNA DKFZp586C1923, cDNA DKFZp434K1126, DNCH1, DRPLA, ADPRTL2, HSPCA, YWHAZ, AP2A2, GRWD, BICD1, MARK3, FLJ10482, FLJ11588, ELOVL5, EUROIMAGE 29222, and DKFZP564A2416, and
a second group of genes that are expressed at lower levels in Dukes° C tumors than in Dukes' B tumors, wherein the second group consists of FUT4, NDUFB7, ASMT, CASP5, PSMF1, PTGER3, FLB0708, RPS21, KIAA0734, FZD3, EIF1A, NEBL, SRI, KCNJ6, ANKRD5, SSX2, AF098968, DJ971N18.2, HSU79252, SP329, FLJ20420, DT1P1B, cDNA DKFZp434E0528, PTPRA, BS69, CREG, PIGL, ILVBL, PANK2, ATP5J, CGI-01, and UBE2N;
comparing the gene expression level of each of the at least five informative genes in the unknown sample to the average gene expression level of that gene in a plurality of reference samples that are Dukes' B stage to determine if each of the at least five informative genes is expressed at higher or lower levels in the unknown sample relative to the plurality of reference samples; and,
classifying the unknown sample as Dukes' C stage if each of the informative genes selected from the first group is expressed at higher levels in the unknown sample than the average expression of that gene in the plurality of reference samples and if each of the informative genes selected from the second group is expressed at lower levels in the unknown sample than the average expression of that gene in the plurality of reference samples.

2. The method of claim 1 wherein at least twenty informative genes are analyzed at each step.

3. The method of claim 2 wherein the at least twenty informative genes are NKX2H, H2BFJ, PADI2, SPAM1, PHKA1, KIAA1827, PSMF1, ASMT, ARF6, PTGER3, BPAG1, SRI, GTF3C1, KIAA0903, FLJ22596, DT1P1B, DKFZp586C1923, UPLC1, ANKRD5, and KIAA0992.

4. The method of claim 1 wherein the gene expression level of each of the at least five informative genes is determined by hybridization to an array of nucleic acid probes.

5. The method of claim 4 wherein the array of nucleic acid probes comprises SEQ ID NOs 102-321.

6. A method of identifying compounds that inhibit or promote metastasis of a colorectal tumor to the regional lymph nodes comprising:
obtaining a first sample of cells from a colorectal tumor before treatment;
treating the tumor with the compound;
obtaining a second sample of cells from the tumor after treatment;
determining the expression level of at least five informative genes in the first and second samples, wherein the at least five informative genes are selected from the group consisting of H2BFH, H2BFJ, LENEP, SPON1, FUT4, PC, NDUFB7, PADI2, SPAM1, PHKA1, NKX2H, RAB7, ASMT, CASP5, PSMF1, SPN, PTGER3, FLB0708, RPS21, BAIAP3, KIAA0734, DVL2, GRB14, FZD3, SRCAP, GTF3C1, RUNX1, EIF1A, NEBL, SYNGR3, PPL, NPIP, BPAG1, SRI, KCNJ6, ARF6, PNMA1, UPLC1, BAI3, SSX2, FLJ22596, FLJ10251, AF098968, KIAA1827, CLIPR-59, ANKRD5, KIAA0903, KIAA0992, FLB4237, clone HRC00953, cDNA DKFZp434E0528, cDNA DKFZp586C1923, cDNA DKFZp434K1126, PIGL, ILVBL, DNCH1, DRPLA, UBE2N, ADPRTL2, CGI-01, HSPCA, PANK2, ATP5J, YWHAZ, AP2A2, GRWD, BICD1, PTPRA, MARK3, BS69, CREG, FLJ10482, FLJ11588, DJ971N18.2, HSU79252, SP329, FLJ20420, DT1P1B, ELOVL5, EUROIMAGE 29222, and DKFZP564A2416;
determining the direction of change between the expression level of each of the at least five informative gene in the first and second samples;
identifying the compound as a promoter of metastasis if the direction of change for each of the at least five informative genes is:
up and the gene is selected from the group consisting of H2BFH, H2BFJ, LENEP, SPON1, PC, PADI2, SPAM1, PHKA1, NKX2H, RAB7, SPN, BAIAP3, DVL2, GRB14, SRCAP, GTF3C1, RUNX1, SYNGR3, PPL, NPIP, BPAG1, ARF6, PNMA1, UPLC1, BAI3, FLJ22596, FLJ10251, KIAA1827, CLIPR-59, KIAA0903, KIAA0992, FLB4237, clone HRC00953, cDNA DKFZp586C1923, cDNA DKFZp434K1126, DNCH1, DRPLA, ADPRTL2, HSPCA, YWHAZ, AP2A2, GRWD, BICD1, MARK3, FLJ10482, FLJ11588, ELOVL5, EUROIMAGE 29222, and DKFZP564A2416; or
down and the gene is selected from the group consisting of FUT4, NDUFB7, ASMT, CASP5, PSMF1, PTGER3, FLB0708, RPS21, KIAA0734, FZD3, EIF1A, NEBL, SRI, KCNJ6, ANKRD5, SSX2, AF098968, DJ971N18.2, HSU79252, SP329, FLJ20420, DT1P1B, cDNA DKFZp434E0528, PTPRA, BS69, CREG, PIGL, ILVBL, PANK2, ATP5J, CGI-01, and UBE2N;or,
identifying the compound as an inhibitor of metastasis if the direction of change for each of the at least five informative genes is:
down and the gene is selected from the group consisting of H2BFH, H2BFJ, LENEP, SPON1, PC, PADI2, SPAM1, PHKA1, NKX2H, RAB7, SPN, BAIAP3, DVL2, GRB14, SRCAP, GTF3C1, RUNX1, SYNGR3, PPL, NPIP, BPAG1, ARF6, PNMA1, UPLC1, BAI3, FLJ22596, FLJ10251, KIAA1827, CLIPR-59, KIAA0903, KIAA0992, FLB4237, clone HRC00953, cDNA DKFZp586C1923, cDNA DKFZp434K1126, DNCH1, DRPLA, ADPRTL2, HSPCA, YWHAZ, AP2A2, GRWD, BICD1, MARK3, FLJ10482, FLJ11588, ELOVL5, EUROIMAGE 29222, and DKFZP564A2416; or
up and the gene is selected from the group consisting of FUT4, NDUFB7, ASMT, CASP5, PSMF1, PTGER3, FLB0708, RPS21, KIAA0734, FZD3, EIF1A, NEBL, SRI, KCNJ6, ANKRD5, SSX2, AF098968, DJ971N18.2, HSU79252, SP329, FLJ20420, DT1P1B, cDNA DKFZp434E0528, PTPRA, BS69, CREG, PIGL, ILVBL, PANK2, ATP5J, CGI-01, and UBE2N.

7. The method of claim 6 wherein at least twenty informative genes are analyzed at each step.

8. The method of claim 7 wherein the at least twenty informative genes are NKX2H, H2BFJ, PADI2, SPAM1, PHKA1, KIAA1827, PSMF1, ASMT, ARF6, PTGER3, BPAG1, SRI, GTF3C1, KIAA0903, FLJ22596, DT1P1B, DKFZp586C1923, UPLC1, ANKRD5, and KIAA0992..

9. The method of claim 6 wherein gene expression level is determined using an array of nucleic acid probes.

10. A method of predicting the efficacy of a compound for treating a colorectal tumor comprising the steps of:
obtaining a first sample of cells derived from a colorectal tumor before treatment with the compound and a second sample of cells derived from the same tumor after treatment;
determining a gene expression level for at least five informative genes in the first and second sample, wherein informative genes are selected from the group consisting of H2BFH, H2BFJ, LENEP, SPON1, FUT4, PC, NDUFB7, PADI2, SPAM1, PHKA1, NKX2H, RAB7, ASMT, CASP5, PSMF1, SPN, PTGER3, FLB0708, RPS21, BAIAP3, KIAA0734, DVL2, GRB14, FZD3, SRCAP, GTF3C1, RUNX1, EIF1A, NEBL, SYNGR3, PPL, NPIP, BPAG1, SRI, KCNJ6, ARF6, PNMA1, UPLC1, BAI3, SSX2, FLJ22596, FLJ10251, AF098968, KIAA1827, CLIPR-59, ANKRD5, KIAA0903, KIAA0992, FLB4237, clone HRC00953, cDNA DKFZp434E0528, cDNA DKFZp586C1923, cDNA DKFZp434K1126, PIGL, ILVBL, DNCH1, DRPLA, UBE2N, ADPRTL2, CGI-01, HSPCA, PANK2, ATP5J, YWHAZ, AP2A2, GRWD, BICD1, PTPRA, MARK3, BS69, CREG, FLJ10482, FLJ11588, DJ971N18.2, HSU79252, SP329, FLJ20420, DT1P1B, ELOVL5, EUROIMAGE 29222, and DKFZP564A2416;
comparing the gene expression level of each of the at least five informative gene in the first and second samples to determine a direction of change in expression from the first to the second sample for each gene, wherein the direction is either up or down;
comparing the direction of change for each gene to the direction of change for that gene from a sample of a first known stage to a sample of a second known stage, wherein the first stage is more advanced than the second stage; and
identifying the compound as effective if the direction of change for each of the at least five informative genes is the same.

11. The method of claim 10 wherein gene expression levels are determined using an array of nucleic acid probes.

12. The method of claim 10 wherein at least twenty informative genes are analyzed at each step.

13. The method of claim 12 wherein the at least twenty informative genes are NKX2H, H2BFJ, PADI2, SPAM1, PHKA1, KIAA1827, PSMF1, ASMT, ARF6, PTGER3, BPAG1, SRI, GTF3C1, KIAA0903, FLJ22596, DT1P1B, DKFZp586C1923, UPLC1, ANKRD5, and KIAA0992.

14. A method of screening to identify test substances which induce or repress expression of genes which are induced or repressed in a colorectal tumor sample that has metastasized compared to a colorectal tumor sample that has not metastasized, comprising:
contacting a colorectal tumor cell with a test substance;
monitoring expression of a transcript or its translation product wherein the transcript is from a gene selected from the group consisting of H2BFH, H2BFJ, LENEP, SPON1, FUT4, PC, NDUFB7, PADI2, SPAM1, PHKA1, NKX2H, RAB7, ASMT, CASP5, PSMF1, SPN, PTGER3, FLB0708, RPS21, BAIAP3, KIAA0734, DVL2, GRB14, FZD3, SRCAP, GTF3C1, RUNX1, EIF1A, NEBL, SYNGR3, PPL, NPIP, BPAG1, SRI, KCNJ6, ARF6, PNMA1, UPLC1, BAI3, SSX2, FLJ22596, FLJ10251, AF098968, KIAA1827, CLIPR-59, ANKRD5, KIAA0903, KIAA0992, FLB4237, clone HRC00953, cDNA DKFZp434E0528, cDNA DKFZp586C1923, cDNA DKFZp434K1126, PIGL, ILVBL, DNCH1, DRPLA, UBE2N, ADPRTL2, CGI-01, HSPCA, PANK2, ATP5J, YWHAZ, AP2A2, GRWD, BICD1, PTPRA, MARK3, BS69, CREG, FLJ10482, FLJ1588, DJ971N18.2, HSU79252, SP329, FLJ20420, DT1P1B, ELOVL5, EUROIMAGE 29222, and DKFZP564A2416;.

15. A method of distinguishing between a Dukes' B and a Dukes' C stage tumor comprising: monitoring the expression level of any five or more genes selected from the group consisting of H2BFH, H2BFJ, LENEP, SPON1, FUT4, PC, NDUFB7, PADI2, SPAM1, PHKA1, NKX2H, RAB7, ASMT, CASP5, PSMF1, SPN, PTGER3, FLB0708, RPS21, BAIAP3, KIAA0734, DVL2, GRB14, FZD3, SRCAP, GTF3C1, RUNX1, EIF1A, NEBL, SYNGR3, PPL, NPIP, BPAG1, SRI, KCNJ6, ARF6, PNMA1, UPLC1, BAI3, SSX2, FLJ22596, FLJ10251, AF098968, KIAA1827, CLIPR-59, ANKRD5, KIAA0903, KIAA0992, FLB4237, clone HRC00953, cDNA DKFZp434E0528, cDNA DKFZp586C1923, cDNA DKFZp434K1126, PIGL, ILVBL, DNCH1, DRPLA, UBE2N, ADPRTL2, CGI-01, HSPCA, PANK2, ATP5J, YWHAZ, AP2A2, GRWD, BICD1, PTPRA, MARK3, BS69, CREG, FLJ10482, FLJ11588, DJ971N18.2, HSU79252, SP329, FLJ20420, DT1P1B, ELOVL5, EUROIMAGE 29222, and DKFZP564A2416; and comparing the expression levels to a database of expression levels of the five or more genes in Dukes' B and Dukes' C stage tumors.

16. The method of claim 15 wherein the expression level of at least twenty genes are monitored and compared.

17. The method of claim 16 wherein the at least twenty genes are NKX2H, H2BFJ, PADI2, SPAM1, PHKA1, KIAA1827, PSMF1, ASMT, ARF6, PTGER3, BPAG1, SRI, GTF3C1, KIAA0903, FLJ22596, DT1P1B, DKFZp586C1923, UPLC1, ANKRD5, and KIAA0992.

18. A method of classifying a colorectal tumor sample as being positive for regional lymph node metastases comprising:
isolating a nucleic acid sample from the colorectal tumor sample;
determining the expression level of at least five informative genes in the sample, wherein informative genes are selected from the group consisting of H2BFH, H2BFJ, LENEP, SPON1, FUT4, PC, NDUFB7, PADI2, SPAM1, PHKA1, NKX2H, RAB7, ASMT, CASP5, PSMF1, SPN, PTGER3, FLB0708, RPS21, BAIAP3, KIAA0734, DVL2, GRB14, FZD3, SRCAP, GTF3C1, RUNX1, EIF1A, NEBL, SYNGR3, PPL, NPIP, BPAG1, SRI, KCNJ6, ARF6, PNMA1, UPLC1, BAI3, SSX2, FLJ22596, FLJ10251, AF098968, KIAA1827, CLIPR-59, ANKRD5, KIAA0903, KIAA0992, FLB4237, clone HRC00953, cDNA DKFZp434E0528, cDNA DKFZp586C1923, cDNA DKFZp434K1126, PIGL, ILVBL, DNCH1, DRPLA, UBE2N, ADPRTL2, CGI-01, HSPCA, PANK2, ATP5J, YWHAZ, AP2A2, GRWD, BICD1, PTPRA, MARK3, BS69, CREG, FLJ10482, FLJ11588, DJ971N18.2, HSU79252, SP329, FLJ20420, DT1P1B, ELOVL5, EUROIMAGE 29222, and DKFZP564A2416;
comparing the expression level of the at least five informative genes to the expression level of the same informative genes in at least one reference colorectal tumor sample that is negative for regional lymph node metastases;
determining if the one or more informative gene in the colorectal tumor sample is expressed at a higher level or a lower level relative to the reference; and
classifying the sample as having regional lymph node metastases if the direction of change in the expression of the at least five informative genes in the unknown sample relative to the reference is the same as the direction of change for that gene in Tables 2 and 3.

19. A kit for classifying a colorectal tumor sample as being positive for regional lymph node metastases comprising:
an array of probes wherein each probe is perfectly complementary to a single gene selected from the group of genes consisting of H2BFH, H2BFJ, LENEP, SPON1, FUT4, PC, NDUFB7, PADI2, SPAM1, PHKA1, NKX2H, RAB7, ASMT, CASP5, PSMF1, SPN, PTGER3, FLB0708, RPS21, BAIAP3, KIAA0734, DVL2, GRB14, FZD3, SRCAP, GTF3C1, RUNX1, EIF1A, NEBL, SYNGR3, PPL, NPIP, BPAG1, SRI, KCNJ6, ARF6, PNMA1, UPLC1, BAI3, SSX2, FLJ22596, FLJ10251, AF098968, KIAA1827, CLIPR-59, ANKRD5, KIAA0903, KIAA0992, FLB4237, clone HRC00953, cDNA DKFZp434E0528, cDNA DKFZp586C1923, cDNA DKFZp434K1126, PIGL, ILVBL, DNCH1, DRPLA, UBE2N, ADPRTL2, CGI-01, HSPCA, PANK2, ATP5J, YWHAZ, AP2A2, GRWD, BICD1, PTPRA, MARK3, BS69, CREG, FLJ10482, FLJ11588, DJ971N18.2, HSU79252, SP329, FLJ20420, DT1P1B, ELOVL5, EUROIMAGE 29222, and DKFZP564A2416; and wherein the array comprises at least 2 probes for each gene in the group of genes;
a computer-readable medium having computer-executable instructions for performing a method comprising: comparing the gene expression level of at least three informative genes in an experimental sample to the expression level of the corresponding gene in a plurality of samples of known Dukes' stage wherein the Dukes' stage is selected from Dukes' B and Dukes' C and determining if the experimental sample is Dukes' B or Dukes' C stage; and
a computer-readable medium having a plurality of gene expression level values for each of the genes in the group of genes in a plurality of colorectal tumor samples of known Dukes' stage.
